(19) United States
(12) Patent Application Publication
(10) Pub. No.: US 2025/0152606 A1
(43) Pub. Date: May 15, 2025

(54) INDUCTION OF IRREVERSIBLE INFERTILITY IN FEMALE MAMMALIAN ANIMALS USING A SINGLE PROGESTERONE IMPLANT

(71) Applicants: THE BOARD OF TRUSTEES OF THE UNIVERSITY OF ILLINOIS, Urbana, IL (US); EPIVARA, Champaign, IL (US)

(72) Inventors: CheMyong KO, Champaign, IL (US); Po-Ching LIN, Champaign, IL (US); Chan Jin PARK, Savoy, IL (US)

(73) Assignees: THE BOARD OF TRUSTEES OF THE UNIVERSITY OF ILLINOIS, Urbana, IL (US); EPIVARA, Champaign, IL (US)

(21) Appl. No.: 18/838,623

(22) PCT Filed: Feb. 17, 2023
(86) PCT No.: PCT/US2023/062800

§ 371 Date: Aug. 15, 2024

Related U.S. Application Data
(60) Provisional application No. 63/311,837, filed on Feb. 18, 2022.

(51) Int. Cl. A61K 31/57; A61K 9/00; A61P 15/18

(52) U.S. Cl. CPC A61K 31/57; A61K 9/0024; A61P 15/18

(57) ABSTRACT

The described technology induces sterility in female animals by a single injection of progestin containing implants formulated to elevate progesterone level for an extended period to neonatal animals. The sterilant can be progesterone or its derivatives which can activate progesterone receptors. Such formulation may be achieved by using a biodegradable copolymer that releases progesterone for more than 10 days. The progesterone-containing implant can be injected intramuscularly, subcutaneously, or intraperitoneally. The progesterone released from the implants over an extended period then prevents the development of uterine glands, thereby irreversibly sterilizing the female animals.

### RELATED APPLICATIONS

This application claims priority under 35 U.S.C. § 119 (e) to U.S. Provisional Patent Application No. 63/311,837, filed Feb. 18, 2022, which is incorporated herein by reference.

### BACKGROUND OF THE INVENTION

It has been well documented that repetitive injections of progesterone during the critical stages of postnatal development can inhibit adenogenesis (the development of uterine gland). For example, repetitive injections of neonatal ewe lambs with norgestomet, a potent synthetic 19-progesterone approved by FDA for use in veterinary medicine (Machado and Kesler, Drug Dev Ind Pharm. 1996; 22 (12): 1211-1216), irreversibly inhibits adenogenesis without inhibiting the development of oviduct, cervix, or vagina, suggesting that the progesterone's inhibitory action was limited to the uterus (Gray et al., Mol Reprod Dev. 2000; 57 (1): 67-78).

In most mammalian species, uterine glands begin to develop postnatally, meaning after birth (Gray et al., Biol Reprod. 2001; 65 (5): 1311-23). Normal development of uterine glands is required for achieving and maintaining pregnancy after reaching reproductive maturity (Kelleher et al., Sci Rep 6, 38078, 2016). If uterine gland development does not begin within a certain period after birth, the uterus will become devoid of glands or will have significantly fewer glands, consequently making the animal infertile. This phenomenon indicates that there is a temporal window for the initiation of uterine gland development and that progesterone plays an inhibitory role if maintained above a threshold level during this developmental window.

Accordingly, neonatal treatment of progesterone may be used as a method for permanently sterilizing female animals, which may replace the most widely used method of female sterilization, spay, that surgically removes ovaries and uteri.

### SUMMARY

The technology disclosed herein is a method of sterilizing female animals at a neonatal stage using a single injectable implant that slowly releases progesterone (P4), its derivatives or synthetic hormones which can activate progesterone receptors. The implant can be a pellet, polymer (biodegradable or non-biodegradable), capsule, or any form of device that can release progesterone for an extended period. The implant can be injected intramuscularly, subcutaneously, or intraperitoneally. The progesterone released from the implant then inhibits adenogenesis, thereby irreversibly sterilizing the female animal.

Accordingly, this disclosure provides a composition for a progesterone implant, comprising about 15 wt. % to about 25 wt. % P4 and a biodegradable copolymer, wherein the implant has been made as a form of microsphere.

This disclosure also provides a method for inducing sterility, comprising treating a neonatal female animal with an implant that slowly releases P4, such as the one described above, wherein the implant maintains an elevated serum P4 level in the neonatal female animals for more than 10 days above a normal P4 serum level for a control neonatal female mammal to irreversibly inhibit adenogenesis throughout the natural life span, thereby inducing sterility.

The invention provides for the use of the compositions described herein for use in inducing sterility in a female mammal. The invention also provides for the use of a composition as described herein for the manufacture of a medicament to treat a mammal that has not been spayed. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. Adult mouse uteri lack glands following neonatal P4 treatment during postnatal day (PND) 3-9. Histological sections of adult control uteri (A) showed extensive glands (arrows), whereas uteri exposed to P4 during PND 3-9 (B) lacked glands in adulthood (Cooke, et al., Biol Reprod. 2012; 86 (3): 63), corroborating observations in isolated epithelial whole mounts (A). E, epithelium; S, stroma. Bar=100 μm (A and B).

FIG. 2. Pharmacokinetics of progesterone treatment in neonatal rats. Three-day-old rats were treated with 2.0 mg P4 Silastic capsule, (n=5-7), injected daily with 0.5 mg P4 for 7 days (n=5-9), or vehicle (n=5-9).

FIG. 3A-C. Histology of uterine horns of Control, Oil-P4, and sC-P4 at 7 and 28 weeks of ages. (A) Tissue sections were stained with hematoxylin and eosin. Arrows indicate uterine glands. (B, C) Quantification of uterine gland number was calculated by the number of glands per observed sections. Error bar, SD (n=3). *, significantly different from control (P<0.05, Student's t-test).

FIG. 4. Pharmacokinetic profile showing that 2 mg equivalent amount of P4 in microspheres (prepared in 0.2 mL dH₂O) injected to 3 months-old ovariectomized rats.

FIG. 5. Immunohistological staining results with HOXA2 (uterine gland marker) obtained from 2 months-old control and pSpay-injected rats.

### DETAILED DESCRIPTION

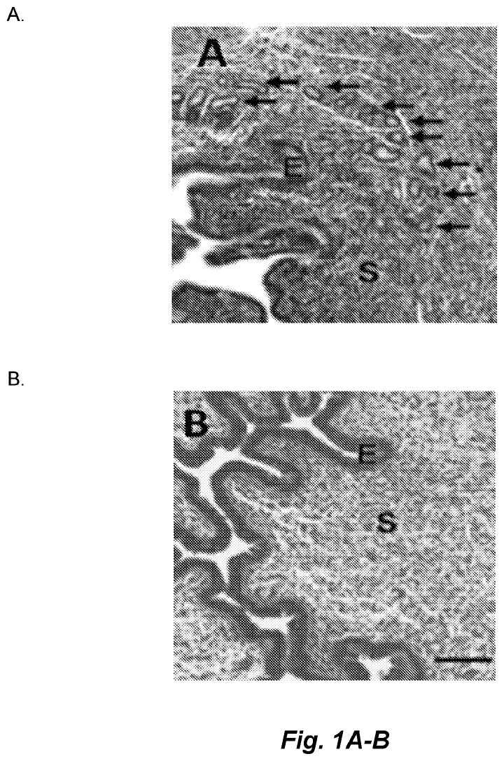

Neonatal mice treated with P4 lacked uterine glands in adulthood. The timing of P4 treatment during adenogenesis is critical for inhibition of uterine gland development. Mice treated with P4 during post-natal days (PND) 3-9, 5-9, or 3-7, but not PND 3-5 or 7-9, abolished adenogenesis at PND 10 (FIG. 1). Importantly, PND 3-9 treated mice lack uterine glands in adulthood, but those treated PND 5-9 or 3-7 showed a resumption of uterine gland development on PND 20 (Cooke, et al., Biol Reprod. 2012; 86 (3): 63).

Cooke et al 2012 disclosed: “Mice were injected daily with P4 (40 μg/g) during PNDs 3-9, 3-5, 3-7, 5-7, 5-9, 7-9, and 10-16 (n=12-20 per group) to encompass the period before, during, and after onset of adenogenesis and peak proliferation of nascent glandular epithelium.”

Taken together, the available data indicates that only extended treatment with P4 during critical uterine gland development permanently inhibits uterine gland development. However, previous studies utilized multiple/daily treatment of P4 or P4-derivatives to inhibit uterine gland development. Therefore, we developed the P4-containing slow-release device and tested it in female rats to achieve a single-shot sterilizing method in female mammals.

Also, another fundamental difference between known methodologies and available products and the technology described herein is that our method inhibits the development of reproductive organs at a very early stage of their development causing irreversible infertility rather than a temporary one. It shuts down the development of the uterine glands and ensures complete and permanent sterility. In contrast, other approaches target adult reproductive organs that are functionally active. One is reminded that the reproductive system is the core component of the body that ensures repetition of life, and therefore is extremely resilient to any external challenge that it may encounter. Notably, the early developmental stage of life is a vulnerable time period, and an alteration of the developing organs at such a critical stage can lead to permanent loss of fertility. We are targeting this moment, with a precision science of reproductive biology, to irreversibly stop uterine gland development, ensuring sterility. Our data proves the disclosed technology herein ensures 100% efficacy in sterilizing females in our model animals, without developing any sign of undesirable side effects.

### Definitions.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure includes methods of making the compounds and compositions of the active ingredients disclosed herein and any analogs or derivatives thereof. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

Progestogens are a class of steroid hormones that bind to and activate the progesterone receptors. One progestogen is progesterone (P4), which is recited herein as the progestogen used in various embodiments of the invention. In other embodiments, the progesterone can be replaced by an amount of one or more other progestogens. Examples of such progestogens include, but are not limited to, 16α-hydroxyprogesterone (16α-OHP), 17α-hydroxyprogesterone (17α-OHP), the 17α-hydroxyprogesterone derivative medroxyprogesterone acetate, the 19-nortestosterone derivative norethisterone, 20α-dihydroprogesterone (20α-DHP), 20β-dihydroprogesterone (20β-DHP), 5α-dihydroprogesterone (5α-DHP), 5β-dihydroprogesterone (5β-DHP), 3β-dihydroprogesterone (3β-DHP), 11-deoxycorticosterone (DOC), and 5α-dihydrodeoxycorticosterone (5α-DHDOC).

The normal P4 serum level for a control neonatal female mammal refers to the average P4 serum level for a particular species of mammal at a particular age. In this context, a control neonatal female mammal refers to a neonatal female mammal having an average P4 serum level with respect to a group of females (e.g., a group of 3-5, 5-10, 10-20, or more, normal neonatal females) of that species of approximately the same age, as would be readily understood by one of skill in the art. With respect to neonatal female mammals, the “same age” refers to individual neonatal female mammals with similar reproductive development. Thus, ‘same age’ can refer to individual female mammals that are approximately the same number of days old or approximately the same number of weeks old (e.g., with some minor variation), depending on the species of mammal. As would be recognized by one of skill in the art, the specific number of days or weeks can be a range, provided the female mammals have similar reproductive development and substantially similar P4 serum levels.

The term “pSpay” refers to the P4-containing microspheres designed for sterilizing female animals as described herein.

### Embodiments of the Technology

This disclosure provides a composition for a slow-release formulation, comprising progesterone (P4) or its analog or derivatives, and a biodegradable polymer or biodegradable copolymer, and the slow-release formulation has been formed into a microsphere. In some embodiments, the biodegradable polymer comprises polylactic-co-glycolic acid (PLGA).

Additionally, this disclosure provides a composition, such as the one described above, for a slow-release formulation, comprising a polydimethylsiloxane capsule that encapsulates progesterone (P4) or a derivative of P4. In some embodiments, the dimensions of the polydimethylsiloxane capsule are about 0.5 mm to about 1.5 mm for an inner diameter (i.d.), about 1.5 mm to about 2.5 mm for an outer diameter (o.d.), and about 2 mm to about 10 mm for a length.

In various embodiments, the polymer or copolymer is crosslinked. In various embodiments, the slow-release formulation of an active ingredient can be formulated by any known means of formulation for slow release, or any known device for slowly releasing an active ingredient. In various embodiments, the slow-release formulation or device for slowly releasing an active ingredient can be implanted into a subject by any known method.

In various embodiments, the slow-release formulation, e.g., the disclosed microsphere or capsule, comprises about 15 wt. % to about 25 wt. % P4. In various other embodiments, the slow-release formulation (microsphere or capsule) comprises about 1 mg to about 50 mg of P4.

This disclosure also provides a method for inducing sterility, comprising:
a) optionally measuring the average normal (baseline) progesterone (P4) level in a control group of one or more naive (untreated) neonatal female animals of about the same age; and
b) implanting a slow-release formulation of progesterone (P4) into a neonatal female mammal, wherein the slow-release formulation maintains an elevated serum P4 level of at least 20 ng/ml higher, or 4 to 8 fold higher than the normal level in the neonatal female mammal for more than 10 days to permanently inhibit uterine gland development throughout the natural life span of the neonatal female mammal, thereby inducing permanent sterility,
optionally wherein the normal level was measured in the control group, and the age of the control group is about the same age as the neonatal female mammal implanted with the slow-release formulation of P4,
optionally, wherein the composition of the slow-release formulation is disclosed herein.

Additionally, this disclosure provides a method for inducing permanent sterility in an animal comprising subcutaneously administering to said animal an effective amount of progesterone (P4) so as to render the animal permanently sterile,
wherein said effective amount of P4 is 1 mg/kg up to about 100 mg/kg,
wherein the P4 is formulated in a slow-release formulation, wherein the sustained release formulation comprises a polymer, wherein theP4 is released over a period of weeks,
wherein the P4 is administered to the animal prior to puberty,
   wherein, optionally, the animal is a dog or cat,

In various embodiments, the age of the neonatal female mammal is 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, or until about the time when the animal reaches puberty.

In various embodiments, the elevated serum P4 level is greater than the normal P4 serum level by about 2-fold, 3-fold, 4-fold, 5-fold, 6-fold, 7-fold, 8-fold, 9-fold, or about 10-fold. In some embodiments, the elevated serum P4 level is greater than the normal P4 serum level by about 4-fold to about 8-fold.

In various embodiments, the elevated serum P4 level is maintained at about 1 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, or about 50 ng/ml or more.

In various embodiments, the elevated serum P4 level in the neonatal female mammal is maintained for more than 10 days. In some embodiments, the elevated serum P4 level in the neonatal female mammal is maintained for about 10 days to about 30 days. In certain embodiments, the elevated serum P4 level in the neonatal female mammal is maintained for about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 25 days, about 30 days, or about 40 or more days.

In various embodiments, the slow-release formulation comprises about 15 wt. % to about 25 wt. % P4. In some embodiments, the slow-release formulation comprises a wt. % of P4 of about 1 wt. %, about 3 wt. %, about 6 wt. %, about 10 wt. %, about 12 wt. %, about 15 wt. %, about 16 wt. %, about 17 wt. %, about 18 wt. %, about 19 wt. %, about 20 wt. %, about 25 wt. %, about 25 wt. %, about 35 wt. %, about 45 wt. %, about 55 wt. %, about 65 wt. %, about 75 wt. %, about 85 wt. %, or about 90 wt. % or more.

In various embodiments, the slow-release formulation comprises about 1 mg to about 50 mg of P4. In some embodiments, the slow-release formulation comprises mg amount of P4 of about 0.2 mg, about 0.5 mg, about 1 mg, about 1.5 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 15 mg, about 20 mg, about 25 mg about 30 mg, about 35 mg, about 40 mg, about 50 mg, about 75 mg, or about 100 mg or more.

In various embodiments, the slow-release formulation comprises P4 and a biodegradable polymer and/or a co-polymer, and the slow-release formulation has been formed into a microsphere or microspheres (a micropowder). In some embodiments, the biodegradable polymer comprises a co-polymer such as polylactic-co-glycolic acid (PLGA). In certain embodiments, a copolymer can comprise random or block copolymers. In various embodiments, the microspheres have an average diameter of about 0.01 microns to about 1000 microns. In other embodiments, the average diameter of the microspheres or micropowder in microns is about 0.01, 0.05, 0.1, 0.2, 0.5, 1.0, 5.0, 10, 15, 20, 25, 50, 75, 100, 150, 200, 250, 300, 350, 400, 450, 500, 600, 700, 800, 900, or 1000 or more microns.

In various embodiments, the microspheres are mixed with an aqueous solution, such as water or buffered water, and the mixture is implanted, injected subcutaneously or intraperitoneally into the neonatal female mammal.

In various embodiments, the slow-release formulation comprises P4 and a polydimethylsiloxane Silastic capsule. In some embodiments, the P4 is encased in the Silastic capsule and the dimensions of the Silastic capsule are about 0.5 mm to about 1.5 mm i.d., about 1.5 mm to about 2.5 mm o.d., and about 2 mm to about 10 mm in length. In some embodiments, the dimensions of the Silastic capsule are i.d. 1.02 mm, o.d. 2.16 mm, length 8 mm. In certain embodiments, the capsule is implanted subcutaneously into the neonatal female mammal.

### STATEMENTS OF THE INVENTION

1. A method for inducing sterility, comprising implanting a slow-release formulation of progesterone (P4) or an analog or derivative thereof into a neonatal female mammal, wherein the slow-release formulation maintains an elevated serum P4 level in the neonatal female mammal for more than 10 days above a normal P4 serum level for a control neonatal female mammal to permanently inhibit uterine gland development throughout the natural life span of the neonatal female mammal, thereby inducing sterility.

2. The method of statement 1 wherein the neonatal female mammal is about 2 days to about 30 days of age, wherein the age depends on the species to be treated.

3. The method of statement 1 or 2 wherein the elevated serum P4 level is greater than the normal P4 serum level by about 2-fold or more.

4 The method of any one of statements 1-3 wherein the elevated serum P4 level is greater than the normal P4 serum level by about 4-fold to about 8-fold.

5. The method of any one of statements 14 wherein the elevated serum P4 level is maintained at about 10 ng/ml to about 50 ng/ml, wherein the age depends on the species to be treated.

6 The method of any one of statements 1-5 wherein the elevated serum P4 level in the neonatal female mammal is maintained for more than 10 days.

7. The method of any one of statements 1-6 wherein the elevated serum P4 level in the neonatal female mammal is maintained for about 10 days to about 30 days, wherein the age depends on the species to be treated.

8. The method of any one of statements 1-7 wherein the slow-release formulation comprises about 10 wt. % to about 97 wt. % P4; or about 15 wt. % to about 25 wt. % P4; wherein the wt. % depends on a matrix that is used in the formulation.

9. The method of any one of statements 1-8 wherein the slow-release formulation comprises about 1 mg to about 50 mg of P4.

10. The method of any one of statements 1-9 wherein the slow-release formulation comprises P4 and a biodegradable polymer that has been formed into microspheres.

11. The method of any one of statements 1-10 wherein the wherein the biodegradable polymer comprises polylactic-co-glycolic acid (PLGA).

12. The method of any one of statements 1-11 wherein the microspheres are mixed with an aqueous solution and the mixture is injected subcutaneously into the neonatal female mammal.

13. The method of any one of statements 1-12 wherein the slow-release formulation comprises P4 and a polydimethylsiloxane (PDMS) Silastic capsule; or comprises a PDMS capsule containing P4.

14. The method of any one of statements 1-13 wherein the P4 is encased in the Silastic capsule and the dimensions of the Silastic capsule are about 0.5 mm to about 1.5 mm i.d., about 1.5 mm to about 2.5 mm o.d., and about 2 mm to about 10 mm in length.

15. The method of any one of statements 1-14 wherein the capsule is implanted subcutaneously into the neonatal female mammal.

### Results.

We developed a prototype product that can sterilize females in essentially any mammalian species when implanted neonatally under the skin (subcutaneously, sc). The implant is made of silicone and releases progesterone (P4) for 2-3 weeks, which inhibits the development of glands in the uterus. Uterine glands play an essential role in achieving and maintaining pregnancy. Hence, inhibition of uterine gland development results in permanent infertility. One caveat in commercialization of this product is that it has to be inserted sc via an incision made on the skin. To overcome this problem and make an injectable alternative, we fabricated P4-releasing microspheres using poly lactic-co-glycolic acid (PLGA). Preliminary study showed that injection of 2 mg P4 to neonatal rats via the microspheres kept serum P4 at a supra physiological level for >2 weeks and completely inhibited uterine gland development as determined by uterine histology. The rats that were injected with P4-microspheres are expected to be sterile as there was absolutely no gland in their uteri. PLGA is a bio-degradable polymer and therefore leaves nothing in the animal after releasing P4 for the intended period. We named P4-microspheres as “pSpay” because the composition is designed in a way that the progesterone released from the microspheres hormonally spay animals. In this disclosure, we extend the data shown for rats and determine the pharmacokinetics and efficacy of the pSpay in other animals.

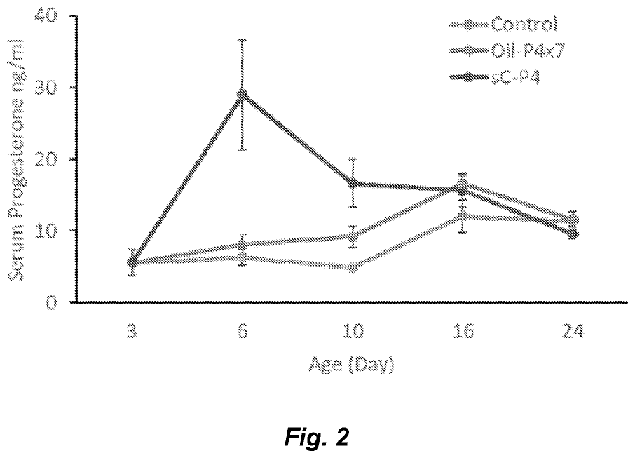

P4 Silastic capsule implant extends P4 exposure in neonatal rats. Serum P4 in the Control rat was <10.0 ng/ml from the start of the study on day 3 until day 10 then increased to approximately 12 ng/ml and remained through day 24. In the sC-P4 group, serum P4 level rose sharply, peaking at 30 ng/ml on day 6, then steadily decreased to control levels on day 16 (FIG. 2). Serum P4 in the Oil-P4 group increased slightly over control, coming down to baseline by day 24.

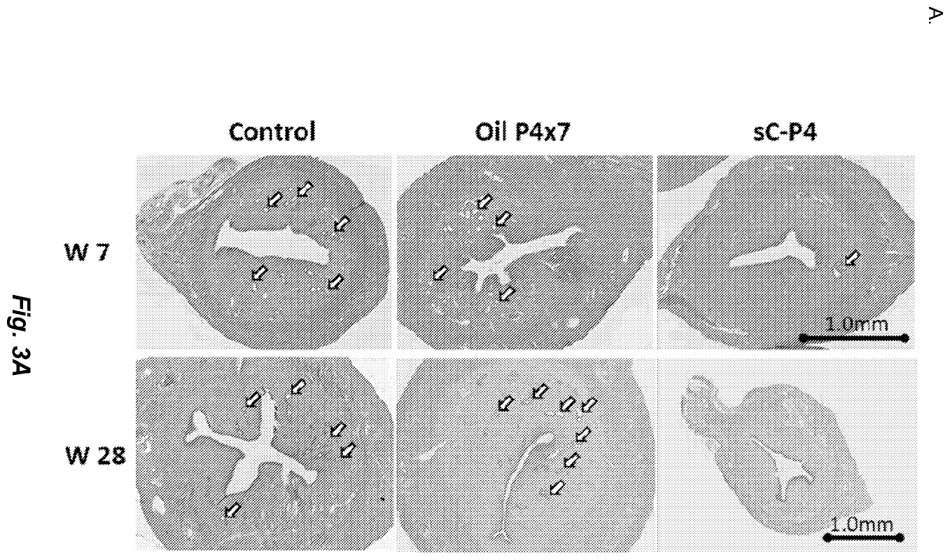

Neonatal P4 Silastic capsule implant permanently inhibits uterine gland development. Uterus sections at weeks 7 and 28 are presented in FIG. 3A. At 7 weeks, control rats showed normal development of uterus and uterine glands (7.7±0.2 glands per section). However, Oil-P4 (4.3±0.4 glands per sections) and sC-P4 (6.8±0.8 glands per sections) groups revealed significantly decreased number of uterine glands (FIG. 3B). At 28 weeks, Control rats showed normal development of uterus and uterine glands (6.8±0.4 glands per sections). The number of uterine glands of Oil-P4 (5.3±0.6 glands per sections) at 28 weeks was not significantly different from that of control. However, sC-P4 (1.1±0.1 glands per sections) group revealed significantly decreased number of uterine glands (FIG. 3C).

Neonatal P4 Silastic capsule implant induces permanent infertility. Starting at 5-months of age, the estrous cycle was determined by microscopy of vaginal smears for 10 days (Table 1). The control group showed a normal estrus-diestrus cycle during the observation. Oil-P4 and sC-P4 also showed a normal estrous cycle. After the estrous cycle observation, female rats were caged with fertility-proven male rats for 1 month to evaluate fertility. Seventy-five percent (¾) of the Control untreated and Oil-P4 groups were fertile, averaging 12 and 13 pups, respectively. By comparison, none of the sC-P4 was fertile.

TABLE 1
Cyclicity and fertility of P4 treated neonatal rats.

| Treatment | Age (months) | n | P4 dose | Cyclicity (%) |
|---|---|---|---|---|
| Oil | 5 | 4 | 0 | 100 |
| Oil-P4 | 5 | 2 | 0.5 mg x7 | 100 |
| sC-P4 | 5 | 3 | 2 mg | 100 |
| Treatment | Age (months) | n | Fertility | Pups/litter |
| Oil | 5 | 4 | 75 | 13 |
| Oil-P4 | 5 | 4 | 75 | 12 |
| sC-P4 | 5 | 4 | 0 | 0 |

### Discussion

The sC-P4 implant achieved >4-fold elevation of serum P4 over Control untreated after 3-days (until experimental day 6), and sC-P4 maintained significantly elevated serum P4 for 13 days (experimental day 16). However, repeated daily injections of P4 only led to a maximum 2-fold increase in serum P4 levels compared to the Control 6 days after the start of P4 injections and serum P4 remained significantly elevated for 10 days (experimental day 16). These results suggest that sC-P4 provides a more sustained and longer-lasting supply of P4 to animals compared to repeated daily injections.

Both Oil-P4 and sC-P4 efficiently suppressed uterine gland development until 7 weeks of age; however, the Oil-P4 group revealed recovered uterine gland structure at 23 weeks. Meanwhile, the sC-P4 group showed a significantly decreased number of uterine glands until 23 weeks. These results suggest that sC-P4 provides sufficient levels of P4 during the neonatal period to inhibit uterine gland development for a longer duration than Oil-P4.

Fertility testing indicates that P4 needs to remain elevated to a high-enough level for an extended amount of time to result in complete fertility. Implantation of sC-P4 resulted in complete sterility. Injection of P4 for 7 days was insufficient to achieve infertility in rats, possibly due to the relatively low levels of sustained progestogen over time. Fertility in all groups correlated with uterine gland development. This technology demonstrates that long-term (>10 days in rats) and sufficient (20-30 ng/ml in rats) treatment of P4 or, its equivalents, suppress the uterine gland development in neonatal mammalian, thereby inducing permanent infertility.

When the experiment was repeated using another type of slow-releasing device (P4-containing microsphere, made of PLGA polymer), the rats exhibited the same reproductive traits (inhibition of uterine gland development and infertility) as were treated with sC-P4, demonstrating that regardless of the types of devices/methodologies used, the slow-release of progesterone for 1-2 weeks is critical for achieving sterility in the subject animals.

### Pharmaceutical Formulations.

The compounds of the formulas described herein can be formulated as pharmaceutical compositions and administered to a mammalian host, such as a canine or feline, in a variety of forms. The forms can be specifically adapted to a chosen route of administration, e.g., by intramuscular, intraperitoneal, or subcutaneous routes.

The active compound may be administered intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

The compounds described herein can be used to prepare pharmaceutical compositions, for example, by combining the compounds with a pharmaceutically acceptable diluent, excipient, or carrier. Some compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium or magnesium) salts of carboxylic acids can also be prepared by analogous methods.

In one embodiment, progestogen (e.g., progesterone) compositions for injectable administration can be in the form of oleaginous suspensions, including oil, such as vegetable oil (e.g., corn oil), cottonseed oil, peanut oil, and/or sesame oil. Other carriers or fillers can be used instead of, or in addition to, oil. Carriers/fillers can include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol. Suspensions can be formulated according to methods available to the art for dispersing and suspending ingredients.

In another embodiment, the compounds or composition described herein can be encapsulated for administration. A capsule can be formed from silicone tubing with plugs at each end to contain a mixture of, for example, a progestogen and oil. The capsules can be placed, such as by injection, in the body of the subject. The progestogen compositions described herein can be formulated for immediate release or in a time release formulation (e.g., slow release). For example, a progestogen can be prepared with carriers that protect the progestogen against rapid release to provide a controlled release formulation.

Many methods for the preparation of controlled/slow-release formulations are known to those skilled in the art. For example, techniques for formulating a variety of sustained- or controlled-delivery means, such as liposome carriers, polymers (e.g., ethylene vinyl acetate, polyanhydrides, silicone, polyglycolic acid, collagen, polyorthoesters, polylactic acid and polylactic, polyglycolic copolymers (PLG)), microparticles, nanoparticles (such as nanospheres, including biodegradable nanospheres or porous beads, and depot injections), a water insoluble polymer and a polyethylene glycol as a water-soluble pore forming agent, or with carrier/matrix such as cholesterol, magnesium stearate, ethyl cellulose N200 carrier/matrix, are known to those skilled in the art. For example, see PCT/US1993/000829 (Supersaxo and Kou), which describes controlled release of porous polymeric microparticles for the delivery of pharmaceutical compositions. Additional examples of sustained-release preparations include semipermeable polymer matrices in the form of shaped articles, e.g., films or microcapsules. Sustained release matrices may include polyesters, hydrogels, polylactides (U.S. Pat. No. 3,773,919 (Boswell), EP0058481B1 (Hutchinson)), copolymers of L-glutamic acid and gamma ethyl-L-glutamate (Sidman et al., Biopolymers, 22:547-556, 1983), poly (2-hydroxyethyl-methacrylate) (Langer et al., J. Biomed. Mater. Res., 15; 167-277, 1981; Langer et al., Chem. Tech. 12:98-105, 1982), ethylene vinyl acetate (Langer et al., supra), or poly-D (−)-3-hydroxybutyric acid (EP0133988A2 (Konig)). Sustained-release compositions also include liposomes, which can be prepared by any of several methods known in the art. See e.g., Epstein et al., Proc. Natl. Acad. Sci. USA, 82:3688-3692, 1985; EP0036676B1; EP0088046B1; and EP0143949B1.

One embodiment provides kits for producing a single-dose administration unit. The kits may contain single and multi-chambered pre-filled syringes containing a progestogen composition and instructions for use (inducing permanent sterility in a mammal).

### Formulation Dosing.

Useful dosages of the compounds or compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg/kg body weight of the recipient per day, preferably in the range of 6 to 90 mg/kg/day, most preferably in the range of 15 to 60 mg/kg/day.

The compound or composition is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

While the compositions described herein are effective for inducing permanent sterility, the compositions are more effective and preferably have fewer side-effects than other known formulations.

The invention thus provides methods for inducing permanent sterility in a mammal by administering to a mammal an effective amount of a compound or composition described herein. A mammal includes a canine, feline, rodent, swine, bovine, ovine, equine, caprine, primate, and the like.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Development of sC-P4 and pSpay. We first used silicone tubing as a P4 delivery device. Briefly, we cut silicone tubing into small pieces into which we inserted P4 dissolved in corn oil. The product was named sC-P4, meaning P4-containing silicone capsule. We then tested sC-P4's efficacy in comparison to a published method and dose (Kelleher et al., 2016). The sC-P4 contained 2 mg P4 and was implanted into 3 days old female rats. When we examined their uteri at the age of 60-80 days, we saw significantly less uterine glands in the sC-P4 implanted rats than control animals. We then tested the impact of sC-P4 implant on fertility when they reached the age of 5 months (when rats display maximum fertility). The results showed that all of the control female rats produced litters (13 pups/litter). However, none of the sC-P4 injected rats produced a litter, indicating complete infertility.

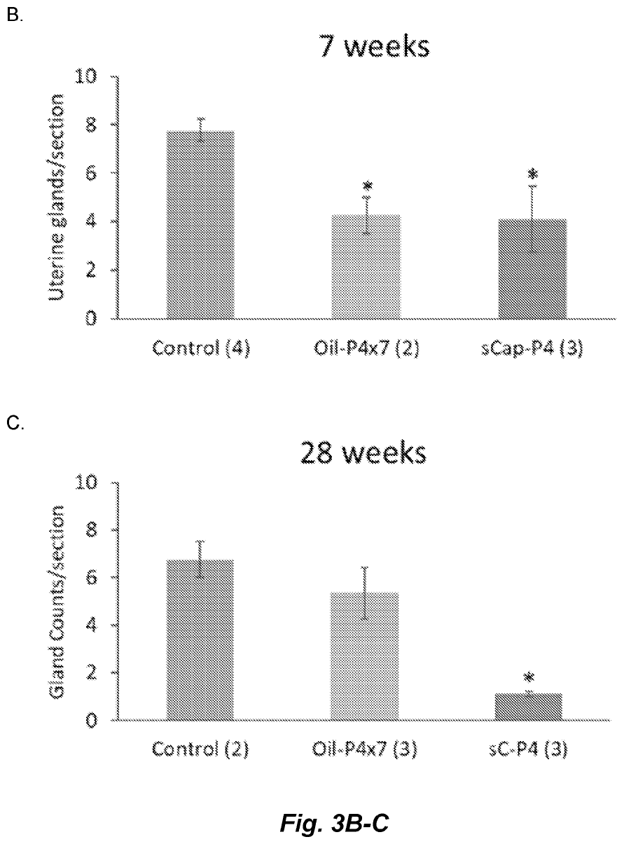

However, sC-P4 carries a limitation for producing them at an industrial level. It is difficult to produce via automation because we have to use tiny silicone tubing, add P4, and seal both ends tight enough so that P4 does not leak out. Further, for use in sterilizing large animals, we may have to use a significantly larger dose than that used in rats. In such case, the tiny tubing will not be able to contain such volume. This reasoning led us to develop a P4-containing microsphere, pSpay. pSpay is synthesized as a form of micropowder and can be produced at a large scale and packaged into a small vial by automation. Furthermore, it has a shelf-life of >1 year. Further, a PK study showed that 2 mg equivalent amount of pSpay (prepared in 0.2 ml dH₂O) injected to 3 months-old ovariectomized rats kept serum P4 levels at supra-physiological levels for >2 weeks (FIG. 4).

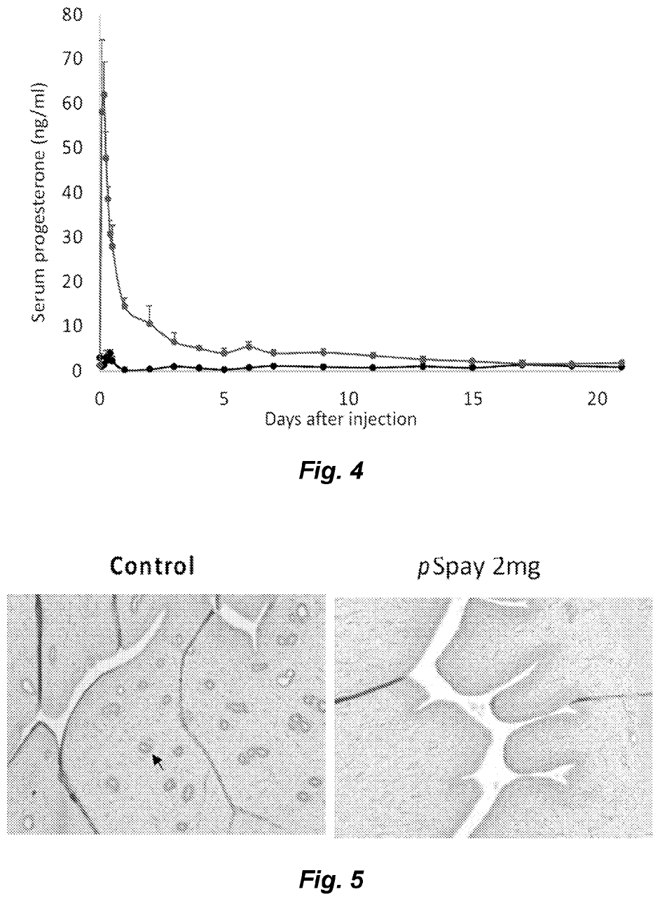

When 2 mg equivalent amount of pSpay was injected subcutaneously to 3 days-old rats, it completely shut down uterine gland development (n=8 for control and n=8 for pSpay groups). Shown in FIG. 5 are immunohistological staining results with HOXA2 (uterine gland marker) obtained from 2 months-old control and pSpay-injected rats; well-developed glands are seen in the control uterus but are absent in the pSpay-injected rat uterus. Because pSpay completely inhibited uterine gland development, these rats are expected to be sterile.

Experiments with Silastic capsules. Neonatal female rats (3-day-old [D3]) were divided into 3 groups for the treatment (Chart 1). The Control group was subcutaneously (sc) injected with sesame oil daily for 7-days. The Oil-P4 group was injected sc with progesterone (P4) (0.5 mg P4/50 μl oil) daily for 7-days. The sC-P4 group was implanted sc with a given single P4 Silastic capsule (sC-P4) (2 mg P4/5 μl oil in a Silastic capsule having dimensions: ID 1.02 mm, OD 2.16 mm, Length 8 mm). Sera were collected by tail clipping (n=5-7) on days 6, 10, 14 and 24, and serum P4 was measured by P4-specific ELISA kit (EIA5486, DRG International Inc., NJ). Uterine histology was examined in weeks 7 and 28. Collected uterine tissues were fixed in 4% paraformaldehyde. Tissues were washed with ethanol, processed, and embedded in paraffin wax. Paraffin-embedded tissue blocks were cut into 6 μm thick sections. For general histological observation, slides were stained with hematoxylin and eosin (HE staining). The number of uterine glands was counted from HE-stained uterine tissue sections (n=2-4). At 5-months-old, the estrous cycle was monitored for 10 days by vaginal cytology (n=2-4). When the rats were in estrous, fertility tests were conducted by introducing fertility-proven males to each female rat (n=4). Data are graphically presented as the mean and standard deviation. Statistical significance of difference in the number of uterine glands in the treated group compared to the control was tested by Student's t-test. Statistical significance was accepted when P-values were lower than 0.05.

CHART 1
Maintaining high P4 levels for greater than 10 days.

| Group | Frequency of dose | Dose |
|---|---|---|
| Intact or oil injected | 7 days | Oil is sesame oil |
| P4 in oil | 7 days | 0.5 mg P4 in 50 μL oil |
| Implant | once on PND3 | 2 mg P4 in 5 μL oil |

### Example 2. Pharmaceutical Dosage Forms

The following formulations illustrate representative pharmaceutical dosage forms that may be used for the administration of a compound or composition described herein, a compound or composition specifically disclosed herein, or a pharmaceutically acceptable salt or solvate thereof (hereinafter referred to as ‘Composition X’):

| (i) Injection 1 (1 mg/mL) | mg/mL |
|---|---|
| ‘Composition (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (ii) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

The invention claimed is:

1. A method for inducing permanent sterility comprising implanting a single slow-release formulation of progesterone (P4) into a neonatal female mammal, wherein the slow-release formulation maintains an elevated serum P4 level in the neonatal female mammal for more than 10 days above a normal P4 serum level for a control neonatal female mammal to permanently inhibit uterine gland development throughout the natural life span of the neonatal female mammal, thereby inducing permanent sterility.

2. The method of claim 1 wherein the neonatal female mammal is about 2 days of age to about 30 days of age.

3. The method of claim 1 wherein the elevated serum P4 level is greater than the normal P4 serum level by about 2-fold or more.

4. The method of claim 1 wherein the elevated serum P4 level is greater than the normal P4 serum level by about 2-fold to about 5-fold.

5. The method of claim 1 wherein the elevated serum P4 level is maintained at about 10 ng/ml to about 50 ng/ml.

6. The method of claim 1 wherein the elevated serum P4 level in the neonatal female mammal is maintained for more than 10 days.

7. The method of claim 1 wherein the elevated serum P4 level in the neonatal female mammal is maintained for about 10 days to about 30 days.

8. The method of claim 1 wherein the slow-release formulation comprises about 10 wt. % to about 97 wt. % P4.

9. The method of claim 1 wherein the slow-release formulation comprises about 1 mg to about 50 mg of P4.

10. The method of claim 1 wherein the slow-release formulation comprises P4 and a biodegradable polymer that has been formed into microspheres.

11. The method of claim 10 wherein the wherein the biodegradable polymer comprises polylactic-co-glycolic acid (PLGA).

12. The method of claim 10 wherein the microspheres are mixed with an aqueous solution and the mixture is injected subcutaneously into the neonatal female mammal.

13. The method of claim 1 wherein the slow-release formulation comprises P4 and a polydimethylsiloxane capsule.

14. The method of claim 13 wherein the P4 is encased in the polydimethylsiloxane capsule and the dimensions of the capsule are about 0.5 mm to about 1.5 mm i.d., about 1.5 mm to about 2.5 mm o.d., and about 2 mm to about 10 mm in length.

15. The method of claim 13 wherein the capsule is implanted subcutaneously into the neonatal female mammal.

16. A method for inducing permanent sterility in a female canine comprising implanting a slow-release formulation of progesterone (P4) into a neonatal female canine, wherein the slow-release formulation maintains an elevated serum P4 level of at least 20 ng/ml in the neonatal female canine for more than 10 days to permanently inhibit uterine gland development throughout the natural life span of the neonatal female canine, thereby inducing permanent sterility.
